(19) United States
(12) Patent Application Publication
(10) Pub. No.: US 2007/0292460 A1
(43) Pub. Date: Dec. 20, 2007

(54) Product release system to atomize non-liquid or highly viscous cosmetic compositions

(76) Inventors: Hartmut Schiemann, Hunfeld (DE); Thomas Krause, Darmstadt (DE); Michael Franzke, Rossdorf (DE); Dirk Weber, Marly (CH); Monika Moenks, Schmitten (CH); Jan Baumeister, Farvagny-le-Grand (CH); Ellen Florig, Grasellenbach (DE)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/471,380

(22) Filed: Jun. 20, 2006

(30) Foreign Application Priority Data
Jun. 20, 2005 (DE) 102005028384.5

(51) Int. Cl. A61K 8/04; A61K 9/12; A61Q 99/00

(52) U.S. Cl. 424/401; 424/43; 424/47

(57) ABSTRACT

A product release system to atomize cosmetic compositions is described, which has (a) pressure-resistant packaging, (b) a capillary-containing spray head, and (c) a propellant-containing cosmetic composition. The atomization is done using the capillary and the composition is non-fluid at 77° F. (25° C.) or has a viscosity greater than 0.0007 lb/in² s (5,000 mPa s). The capillary preferably has a diameter of 0.004-0.04 in (0.1 to 1 mm) and a length of 0.2-4 in (5 to 100 mm). The spray rate is preferably 0.0004-0.2 oz/s (0.01 to 5 g/s). The composition can be, in particular, gel-like, waxy, or emulsion-like and used for the treatment of hair or skin.

### FIELD OF THE INVENTION

The object of the present invention is a product release system for atomizing cosmetic compositions, which has pressure-resistant packaging, a capillary-containing spray head, and a propellant-containing cosmetic composition, and wherein the composition is non-liquid or highly viscous at 77° F. (25° C.). The object of the invention is also the use of the product release system for hair treatment as well as a corresponding method for hair treatment.

### BACKGROUND OF THE INVENTION

Many cosmetic products, particularly cosmetic hair products such as, for example, styling gels, styling creams, hair waxes, or creamy hair-repair products, are present in a non-liquid or highly viscous form. The disadvantages of these types of products, which are normally removed from tubes or jars, are their poor dispensability and distributability on the hair and the associated more extensive and uneven stress on the hair. In addition, these products must be applied with the hands, which makes it necessary to clean them and, in the case of jar products, there is a risk of microbial contamination with multiple use. Spray products provide better dispensing, distribution, and application properties. However, classic non-liquid or gel products with viscosities greater than 0.0007 lb/in² s (5,000 mPa s) can not be atomized with conventional aerosol or nonaerosol spray systems because of their consistency. They either do not provide any spray or the spray pattern is too imprecise with a high percentage of large spray droplets.

In addition, with cosmetic products, consumers want the most pleasant feel possible of the product mass on the skin during the application. Many cosmetic hair products contain additional ingredients, for example, hair-conditioning agents, moisturizers, or anti-dandruff agents. In order to stabilize the additional ingredients, often a higher viscosity or reduced flow capacity is required, whereby the product removal, the applicability, and the even distribution are influenced. Previously, it was not possible to dispense higher viscosity or non-fluid formulations from the packaging with as much precision and even distribution as can be obtained with low-viscosity products. Liquid products with a lower viscosity are easier to extract and easier to distribute; however, these products often do not contain all the desired active ingredients and additives in a stable form and often have a less intensive hair- and scalp-conditioning and hair- or scalp-care effect. In addition, it has been difficult in the past to apply products containing skin ingredients directly and precisely to the scalp.

A process for atomizing liquid is known from WO 03/051523 A1 with which the spray is formed using a capillary. Only the application with respect to atomizing liquid compositions is described. A fixture for atomizing liquid products is described in WO 03/051522 A2, wherein the spray is formed using a capillary. Only the use of liquid compositions for atomizing, which can also be highly viscous, are described, wherein 0.0007 lb/in² s (5,000 mPa s) is mentioned as the maximum sprayable viscosity.

Thus, there is a need for highly viscous or solid cosmetic hair products with improved dispensability, better distributability, and a more pleasant feel on the scalp during application. In doing so, the hair-conditioning and scalp-conditioning effects should correspond to those of highly viscous products, or even go beyond the effects of previously known products.

### SUMMARY OF THE INVENTION

The object of the invention is a product release system for atomizing cosmetic compositions. The product release system has the following features:

(a) pressure-resistant packaging;

(b) a spray head containing a capillary; and

(c) a propellant-containing cosmetic composition,

wherein the atomization is done using the capillary, and the composition is non-liquid at 77° F. (25° C.) and/or has a viscosity greater than 0.0007 lb/in² s (5,000 mPa s) (measured with a HAAKE VT-550 Rheometer, SV-DIN test body at a temperature of 77° F. (25° C.) and a shear speed of 12.9 s⁻¹).

### DETAILED DESCRIPTION OF THE INVENTION

The term “atomize” is understood to mean the release of the product in the form of dissipated particles. The dissipated particles can have varying shapes, consistencies, and sizes. The properties of the atomized particles can include everything from fine aerosol atomized spray to liquid drops, snow-like drops, solid spray flakes, and spray foam.

The quantities of ingredients (e.g., wt. %) indicated in the following are each based on the basic composition without propellant unless explicitly indicated otherwise. The quantities of the propellant are based on the total composition including propellant.

The properties of the compositions to be used according to the invention that are related to consistency are based on the base composition without propellant (unless explicitly indicated otherwise). Non-liquid compositions in terms of the invention are particularly non-flow-capable compositions, which, for example, can be determined due to the fact that they will not flow off of a glass surface tilted at 45° at a temperature of 77° F. (25° C.). Non-liquid compositions can be, for example, solid, pasty, or creamy. Gel compositions are characterized in that the memory module G′ is larger than the loss module G″ at 77° F. (25° C.) with oscillographic measurements in the typical measurement range (0.01 to 40 Hz).

The viscosity of the composition to be used is preferably greater than 5,000 up to 100,000, or especially preferably 0.0010-007lb/in² s (10,000 to 50,000mPa s), and very especially preferably 0.004-0.005 lb/in² s (25,000 to 35,000 mPa s), measured with a HAAKE VT-550 Rheometer, SV-DIN test body at a temperature of 77° F. (25° C.) and a shear speed of 12.9 s⁻¹.

Aerosol spray cans constructed of metal or plastic can be used as the pressure-resistant packaging. Preferred metals are tin plates and aluminum, while the preferred plastic is polyethylene terephthalate.

Suitable spray systems with capillary-containing spray heads, with which the spray is formed using a capillary, are described in WO 03/051523 A1 and in WO 03/051522 A2. The capillaries preferably have a diameter of 0.004-0.04 in (0.1 to 1 mm), or particularly of 0.008-0.02 in (0.2 to 0.6 mm) and a length that is preferably 0.2-4 in (5 to 100 mm), or particularly 0.2-2 in (5 to 50 mm). The spray principle is also described in Aerosol Europe, vol. 13 no. 1-2005, pages 6-11. The spray system is based on the principle of capillary atomization. The conventional swirl nozzle as well as, if necessary, the uptake tube are replaced by capillaries. The energy-consuming and propellant-intensive swirling of the content of the can and the required strong dilution of the product with solvents is not necessary as compared to conventional spray systems. Even if only a small quantity of propellant is used, the product rises upward on the wall of the uptake tube capillary and is propelled, after the valve in the (wider) capillary of the spray head, in the direction of the exit opening. In this manner, small drops from the flowing propellant are torn from the surface of the liquid and continue to flow as aerosol. Since there is no swirl chamber to inhibit the flow of the product nor any atomizing nozzle available, the energy in the system can be used much more efficiently to create the desired spray. The spray rate can be adjusted via the selection of the capillary geometry in conjunction with the interior pressure created by the propellant or a propellant mixture. Preferred spray rates are 0.0004-0.02 oz/s (0.01 to 0.5 g/s), or particularly 0.004-0.01 oz/s (0.1 to 0.3 g/s). The size of the spray drops created with the atomization can be adjusted via the selection of the capillary geometry in conjunction with the interior pressure or the viscosity of the composition. Suitable capillary atomization systems can be obtained in a product called TRUSPRAY® from Boehringer Ingelheim microParts GmbH.

The preferred drop size distributions are those with which the dv(50) value is a maximum of 0.008 in (200 μm), e.g., of from 0.002-0.008 in (50 to 200 μm) with a maximum of 100 μm e.g., of from 0.003-0.004 in (70 to 90 μm) being especially preferred, and/or with which the dv(90) value is a maximum of 0.006 in (160 μm), e.g., of from 0.004-0.006 in (90 to 160 μm), with a maximum of 150 μm e.g., of from 0.005-0.006 in (115 to 150 μm) being especially preferred. The dv(50) or dv(90) values indicate the maximum diameter that 50% or 90% of all droplets have. The drop size distribution can, for example, be determined with the help of a particle measurement unit based on laser beam diffraction, e.g., a Malvern particle sizer measuring device. Compositions that form a snow-like consistency, flakes, or foam (spray foam) upon exiting the capillary spray system are also preferred.

The propellant to be used can be selected from lower alkanes, particularly C3 to C5 hydrocarbons such as, for example, n-butane, i-butane, and propane, or also mixtures thereof, as well as dimethylethers or fluorine hydrocarbons such as F 152a (1,1-difluoroethane) or F 134 (tetrafluoroethane) as well as other gaseous propellants present with the pressures considered, such as, for example, N₂, N₂O, and CO₂ as well as mixtures of the aforementioned propellants. The propellant is preferably selected from propane, n-butane, isobutane, dimethylether, fluorinated hydrocarbons, and mixtures thereof. The content of propellant is, in addition, preferably 15 to 85 wt. %, with 25 to 75 wt. % being especially preferred.

The composition contains cosmetically acceptable solvents, preferably an aqueous, alcoholic, or aqueous alcoholic medium. The lower alcohols with 1 to 4 C atoms, such as ethanol and isopropanol, can be contained as alcohols, particularly those typically used for cosmetic purposes. The composition can be in a pH range of 2.0 to 9.5. A pH range of 4 to 8 is particular preferred, providing no special application forms require other pH values. As additional co-solvents, organic solvents or a mixture of solvents with a boiling point of less than 752° F. (400° C.) can be contained in a quantity of from 0.1 to 15 wt. % or preferably of from 1 to 10 wt. %. Unbranched or branched hydrocarbons such as pentane, hexane, isopentane, and cyclic hydrocarbons such as cyclopentane and cyclohexane are particularly suitable as additional co-solvents. These volatile hydrocarbons can also be used as propellants. Other, especially preferred water-soluble solvents are glycerin, ethylene glycol, and propylene glycol in a quantity of up to 30 wt. %.

The product release system according to the invention can be used for hair treatment. The compositions can be agents for the care of hair such as, for example, hair-repair products or hair rinses, which, for example, can be applied as leave-on or rinse-off products; agents for the temporary reshaping and/or stabilizing of the hairstyle (styling agent), for example hair sprays, hair lacquers, hair gels, hair waxes, styling creams, etc.; permanent, semipermanent, or temporary hair colorants, for example oxidative hair colorants or nonoxidative hair tinting agents or hair bleaching agents; permanent hair restructuring agents, for example in the form of a mildly alkaline or acidic permanent wave or hair straightening agents containing a reducing agent, or in the form of permanent wave fixing agents containing an oxidizing agent.

In one embodiment, the composition to be used according to the invention is gel-like and contains at least one thickener or gel-former preferably in a quantity of from 0.01 to 20 wt. %, or of from 0.1 to 10 wt. %, of from 0.5 to 8 wt. %, or especially preferably of from 1 to 5 wt. %. Materials for which the function “Viscosity Increasing Agent” is indicated in the International Cosmetic Ingredient Dictionary and Handbook, 10th edition, 2004 are essentially suitable. The thickener or gel-former is preferably a thickening polymer and is especially preferably selected from copolymers consisting of at least one first type of monomer, which is selected from acrylic acid and methacrylic acid, and at least one second type of monomer, which is selected from esters of acrylic acid and ethoxylated fatty alcohol; crosslinked polyacrylic acid; crosslinked copolymers consisting of at least one first type of monomer, which is selected from acrylic acid and methacrylic acid, and at least one second type of monomer, which is selected from esters of acrylic acid with C10 to C30 alcohols; copolymers consisting of at least one first type of monomer, which is selected from acrylic acid and methacrylic acid, and at least one second type of monomer, which is selected from esters of itaconic acid and ethoxylated fatty alcohol; copolymers consisting of at least one type of monomer, which is selected from acrylic acid and methacrylic acid, at least one second type of monomer, which is selected from esters of itaconic acid and ethoxylated C10 to C30 alcohol, and a third type of monomer, which is selected from C1 to C4 aminoalkyl acrylates; copolymers consisting of two or more monomers, which are selected from acrylic acid, methacrylic acid, acrylic acid esters and methacrylic acid esters; copolymers consisting of vinyl pyrrolidone and ammonium acryloyl dimethyltaurate; copolymers consisting of ammonium acryloyl dimethyltaurate and monomers selected from esters of methacrylic acid and ethoxylated fatty alcohols; hydroxyethyl cellulose; hydroxypropyl cellulose; hydroxypropyl guar; glyceryl polyacrylate; glyceryl polymethacrylate; copolymers consisting of at least one C2, C3, or C4 alkylene and styrene; polyurethane; hydroxypropyl starch phosphate; polyacrylamide; copolymers crosslinked with decadiene consisting of maleic acid anhydride and methyl vinyl ether; locust bean gum; guar gum; xanthan; dehydroxanthan; carrageenan; karaya gum; hydrolyzed corn starch; copolymers consisting of polyethylene oxide, fatty alcohols, and saturated methylene diphenyl diisocyanate (e.g., PEG-150/stearyl alcohol/SMDI copolymer).

In an additional embodiment, the composition is waxy and contains at least one wax that is solid at 77° F. (25° C.) in a quantity of preferably from 10 to 80 wt. %, particularly of from 20 to 60 wt. %, or of from 25 to 50 wt. %, as well as, if necessary, other water-insoluble materials that are liquid at room temperature. The waxy consistency is preferably characterized in that the needle penetration number (unit of measurement 0.004 in (0.1 mm), test weight 3.5 oz (100 g), testing time 5 s, test temperature 77° F. (25° C.); according to DIN 51 579) preferably ranges from 2 to 70, or particularly from 3 to 40, and/or that the composition can be melted and has a solidification point that is greater than 77° F. (25° C.), or is preferably in a range of from 86-158° F. (30 bis 70° C.), or especially preferably in a range of from 104-131° F. (40 to 55° C.).

Principally any wax that is known in the prior art can be used as a wax or waxy material. These waxes include animal, vegetable, mineral, and synthetic waxes, microcrystalline waxes, macrocrystalline waxes, solid paraffins, petroleum jelly, Vaseline, ozocerite, montan wax, Fischer-Tropsch wax, polyolefin waxes, e.g., polybutene, beeswax, wool wax, and its derivatives such as, for example, wool wax alcohols, candelilla wax, olive wax, carnauba wax, Japan wax, apple wax, hydrogenated fats, fatty acid esters, fatty acid glycerides with a solidification point greater than 104° F. (40° C.), silicone waxes or hydrophilic waxes such as, for example, high-molecular-weight polyethylene glycol waxes with a molecular weight of from 800 to 20,000, preferably of from 2,000 to 10,000 g/mol. The waxes or waxy materials have a solidification point greater than 77° F. (25° C.), or preferably greater than 104° F. (40° C.) or 131° F. (55° C.). The needle penetration number (0.004 in (0.1 mm)-3.5 oz (100 g), 5 s, 77° F. (25° C.) (according to DIN 51 579) preferably lies in the range of from 2 to 70, or especially 3 to 40.

In another embodiment, the composition is emulsion-like, wherein the consistency is preferably creamy. The emulsion can be a water-in-oil emulsion, an oil-in-water emulsion, a microemulsion, or a higher emulsion. In addition to water, preferably at least one hydrophobic oil that is liquid at room temperature (77° F. (25° C.)) as well as at least one emulsifier is contained. The oil content is preferably from 1 to 20 wt. %, particularly from 2 to 10 wt. %. The emulsifier content is preferably from 0.01 to 30 wt. %, or particularly from 0.1 to 20 wt. %, or from 0.5 to 10wt. %.

Suitable liquid, hydrophobic oils have a melting point of less than 77° F. (25° C.) and a boiling point of preferably greater than 482° F. (250° C.), or particularly greater than 572° F. (300° C.). Volatile oils can also be used. In principle, any oil generally known to a person skilled in the art can be used. Suitable oils are vegetable or animal oils, mineral oils (liquid paraffin), silicone oils or their mixtures. Hydrocarbon oils, e.g., paraffin or isoparaffin oils, squalane, oils from fatty acids and polyols, especially triglycerides, are suitable. Suitable vegetable oils are, for example, sunflower oil, coconut oil, castor oil, lanolin oil, jojoba oil, corn oil, soy oil.

Suitable emulsifiers can include nonionic, anionic, cationic, or zwitterionic surfactants. Suitable nonionic surfactants are, for example,
ethoxylated fatty alcohols, fatty acids, fatty acid glycerides, or alkyl phenols, especially addition products of 2 to 30 mol ethylene oxide and/or 1 to 5 mol propylene oxide to C8 to C22 fatty alcohols, to C12 to C22 fatty acids, or to alkyl phenols with 8 to 15 C atoms in the alkyl group;
C12 to C22 fatty acid mono- and diesters of addition products of 1 to 30 mol ethylene oxide to glycerol;
addition products of 5 to 60 mol ethylene oxide to castor oil or hydrogenated castor oil;
fatty acid sugar esters, especially esters from saccharose and one or two C8 to C22 fatty acids, INCI: Sucrose Cocoate, Sucrose Dilaurate, Sucrose Distearate, Sucrose Laurate, Sucrose Myristate, Sucrose Oleate, Sucrose Palmitate, Sucrose Ricinoleate, Sucrose Stearate;
esters from sorbitan and one, two or three C8 to C22 fatty acids and a degree of ethoxylation of 4 to 20;
polyglyceryl fatty acid esters, especially from one, two or more C8 to C22 fatty acids and polyglycerol with preferably 2 to 20 glyceryl units;
alkylglucosides, alkyloligoglucosides, and alkylpolyglucoside with C8 to C22 alkyl groups, e.g., decyl glucoside or lauryl glucoside.

Suitable anionic surfactants are, for example, salts and esters of carboxylic acids, alkyl ether sulfates and alkyl sulfates, fatty alcohol ether sulfates, sulfonic acids and their salts (e.g., sulfosuccinates or fatty acid isethienates), phosphoric acid esters and their salts, acylamino acids and their salts. A comprehensive description of these anionic surfactants is found in the publication “FIEDLER—Lexikon der Hilfsstoffe” [FIEDLER—Dictionary ofAdjuvants], volume 1, fifth edition (2002), pages 97 to 102, to which expressed reference is made. Preferred surfactants are mono-, di-, and/or triesters of phosphoric acid with addition products of from 2 to 30 mol ethylene oxide to C8 to C22 fatty alcohols.

Suitable amphoteric surfactants are, for example, derivatives of aliphatic quaternary ammonium, phosphonium, and sulfonium compounds of the formula 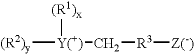
wherein R¹ represents a straight-chain or branched-chain alkyl, alkenyl, or hydroxyalkyl group with 8 to 18 C atoms and 0 to about 10 ethylene oxide units and 0 to 1 glycerol units; Y is an N-, P-, or S-containing group; R² is an alkyl or monohydroxyalkyl group with 1 to 3 C atoms; the total of x+y equals 2 if Y is a sulfur atom, and the total of x+y equals 3 if Y is a nitrogen atom or a phosphorus atom; R³ is an alkylene or hydroxyalkylene group with 1 to 4 C atoms, and Z(−) represents a carboxylate, sulfate, phosphonate, or phosphate group. Other amphoteric surfactants such as betaines are also suitable. Examples of betaines include C8 to C18 alkylbetaines such as cocodimethylcarboxymethylbetaine, lauryldimethylcarboxymethylbetaine, lauryldimethyl-alpha-carboxyethylbetaine, cetyldimethylcarboxymethylbetaine, oleyldimethylgammacarboxypropylbetaine, and lauryl-bis-(2-hydroxypropyl)-alpha-carboxyethylbetaine; C8 to C18 sulfobetaines such as cocodimethylsulfopropylbetaine, stearyldimethylsulfopropylbetaine, lauryldimethylsulfoethylbetaine, lauryl-bis-(2-hydroxyethyl)sulfopropylbetaine; the carboxyl derivatives of imidazole, C8 to C 18 alkyldimethylammonium acetate, C8 to C18 alkyldimethylcarbonylmethylammonium salts, as well as C8 to C18 fatty acid alkylamidobetaines such as, for example, coconut fatty acid amidopropylbetaine and N-coconut fatty acid amidoethyl-N-[2-(carboxymethoxy)ethyl]-glycerin (CTFA name: Cocoamphocarboxyglycinate).

Suitable cationic surfactants contain amino groups or quaternized hydrophilic ammonium groups that carry a positive charge in solution and can be represented by the general formula
N⁽⁺⁾R¹R²R³R⁴ X⁽⁻⁾
wherein R¹ to R⁴, independently from one another, stand for aliphatic groups, aromatic groups, alkoxy groups, polyoxyalkylene groups, alkylamido groups, hydroxyalkyl groups, aryl groups, or alkaryl groups with 1 to 22 C atoms, wherein at least one radical has at least 6, preferably at least 8, C atoms and X⁻ represents an anion, for example a halide, acetate, phosphate, nitrate, or alkyl sulfate, but preferably a chloride. In addition to the carbon atoms and the hydrogen atoms, the aliphatic groups can also contain cross-compounds, or other groups, such as, for example, additional amino groups. Examples of suitable cationic surfactants are the chlorides or bromides of alkyldimethylbenzylammonium salts, alkyltrimethylammonium salts, e.g., cetyltrimethylammonium chloride or bromide, tetradecyltrimethylammonium chloride or bromide, alkyldimethylhydroxyethylammonium chlorides or bromides, dialkyldimethylammonium chlorides or bromides, alkylpyridinium salts, for example lauryl- or cetylpyridinium chloride, alkylamidoethyltrimethylammonium ether sulfates as well as compounds with cationic character such as amine oxides, e.g., alkylmethylamine oxides or alkylaminoethyldimethylamine oxides. Especially preferred are C8-22 alkyldimethylbenzylammonium compounds, C8-22 alkyltrimethylammonium compounds, especially cetyltrimethylammonium chloride, C8-22 alkyldimethylhydroxyethylammonium compounds, di-(C8-22alkyl)-dimethylammonium compounds, C8-22 alkylpyridinium salts, C8-22 alkylamidoethyltrimethylammonium ether sulfates, C8-22 alkylmethylamine oxides, and C8-22 alkylaminoethyldimethylamine oxides.

The cosmetic composition to be used according to the present invention can also contain at least one additional active cosmetic ingredient or additive for the hair or skin/scalp. This active ingredient or additive can, for example, be selected from hair-conditioning materials, hair-setting materials, silicone compounds, photoprotective materials, preservatives, pigments, direct-penetrating hair dyes, particle-shaped materials, oxidizing agents, reducing agents, and oxidative hair colorant precursor products. The active ingredients and additives, depending on the type and intended use, are preferably contained in a quantity of from 0.01 to 20 wt. %, or particularly of from 0.05 to 10 wt. %, or of from 0.1 to 5 wt. %.

In one embodiment, the agent according to the invention, as a hair-care or hair-setting additive, contains at least one polymer with anionic groups or groups that can be ionized preferably in a quantity of from 0.01 to 20 wt. % or of from 0.05 to 10 wt. %, with 0.1 to 5 wt. % being especially preferred. Groups that can be anionized are understood to be acid groups such as, for example, carboxylic acid, sulfonic acid, or phosphoric acid groups that can be deprotonated using typical bases such as, for example, organic amines or alkali- or alkaline earth hydroxides. The anionic polymers can be partially or completely neutralized with an alkaline neutralizing agent. Such types of agents in which the acidic groups are neutralized in the polymer to 50 to 100%, or especially preferably to 70-100%, are preferred. Organic or inorganic bases can be used as the neutralizing agent. Particular examples of bases are amino alkanols such as, for example, aminomethylpropanol (AMP), triethanolamine or monoethanolamine, and also ammonia, NaOH, and KOH among others.

The anionic polymer can be a homo- or copolymer with acid group-containing monomer units derived from natural or synthetic sources, which, if necessary, can be polymerized with comonomers that contain no acid groups. Among the acid groups that can be considered are sulfonic acid, phosphoric acid, and carboxylic acid groups, of which the carboxylic acid groups are preferred. Suitable acid group-containing monomers are, for example, acrylic acid, methacrylic acid, crotonic acid, maleic acid, and maleic anhydride, maleic acid monoesters, especially the C1 to C7 alkyl monoesters of maleic acid, as well as aldehydocarboxylic acids or ketocarboxylic acids. Comonomers that are not substituted with acid groups are, for example, acrylamide, methacrylamide, alkyl- and dialkylacrylamide, alkyl and dialkylmethacrylamide, alkyl acrylate, alkyl methacrylate, vinylcaprolactone, vinylpyrrolidone, vinyl ester, vinyl alcohol, propylene glycol or ethylene glycol, amine-substituted vinyl monomers such as, for example, dialkylaminoalkyl acrylate, dialkylaminoalkyl methacrylate, monoalkylaminoalkyl acrylate, and monoalkylaminoalkyl methacrylate, wherein the alkyl groups of these monomers are preferably C1 to C7 alkyl groups, with C1 to C3 alkyl groups being especially preferred.

Suitable polymers with acid groups are especially homopolymers of acrylic acid or methacrylic acid, copolymers of acrylic acid or methacrylic acid with monomers selected from acrylic acid or methacrylic acid esters, acrylamides, methacrylamides and vinylpyrrolidone, homopolymers of crotonic acid as well as copolymers of crotonic acid with monomers selected from vinyl esters, acrylic acid or methacrylic acid esters, acrylamides and methacrylamides that are uncrosslinked or crosslinked with polyfunctional agents. A suitable natural polymer is, for example, shellac.

Preferred Polymers with Acid Groups are:

Terpolymers from acrylic acid, alkyl acrylate, and N-alkylacrylamide (INCI designation: Acrylate/Acrylamide Copolymer), especially terpolymers from acrylic acid, ethyl acrylate and N-tert-butylacrylamide; crosslinked or uncrosslinked vinyl acetate/crotonic acid copolymers (INCI designation: VA/Crotonate Copolymer); copolymers from one or more C1 to C5 alkyl acrylates, especially C2 to C4 alkyl acrylates and at least one monomer selected from acrylic acid or methacrylic acid (INCI designation: Acrylate Copolymer), e.g., terpolymers from tert-butyl acrylate, ethyl acrylate and methacrylic acid; sodium polystyrenesulfonate; vinylacetate/crotonic acid/vinyl alkanoate copolymers, for example, copolymers from vinyl acetate, crotonic acid and vinyl propionate; copolymers from vinyl acetate, crotonic acid and vinyl neodecanoate (INCI designations: VA/Crotonate/Vinyl Propionate Copolymer, VA/Crotonate/Vinyl Neodecanoate Copolymer); aminomethylpropanol acrylate copolymers; copolymers from vinylpyrrolidone and at least one further monomer selected from acrylic acid and methacrylic acid as well as, if necessary, acrylic acid esters and methacrylic acid esters; copolymers from methyl vinyl ether and maleic acid monoalkylesters (INCI designations: Ethyl Ester of PVM/MA Copolymer, Butyl Ester of PVM/MA Copolymer); aminomethylpropanol salts of copolymers from allyl methacrylate and at least one further monomer selected from acrylic acid, and methacrylic acid as well as, if necessary, acrylic acid esters and methacrylic acid esters; crosslinked copolymers from ethyl acrylate and methacrylic acid; copolymers from vinyl acetate, mono-n-butyl maleate and isobomyl acrylate; copolymers from two or more monomers selected from acrylic acid and methacrylic acid as well as, if necessary, acrylic acid esters and methacrylic acid esters; copolymers from octylacrylamide and at least one monomer selected from acrylic acid and methacrylic acid as well as, if necessary, acrylic acid esters and methacrylic acid esters; polyesters from diglycol, cyclohexanedimethanol, isophthalic acid and sulfoisophthalic acid, wherein the alkyl groups of the aforementioned polymers as a rule preferably possess 1, 2, 3, or 4 C atoms.

In one embodiment, the agent according to the invention, as a hair-conditioning or hair-setting additive, contains at least one zwitterionic and/or amphoteric polymer preferably in a quantity of from 0.01 to 20 wt. % or of from 0.05 to 10 wt. %, or especially preferably of from 0.1 to 5 wt. %. Zwitterionic polymers simultaneously have at least one anionic and at least one cationic charge. Amphoteric polymers exhibit at least one acidic group (e.g., carboxylic acid or sulfonic acid group) and at least one alkaline group (e.g., amino group). Acidic groups can be deprotonated using typical bases such as, for example, organic amines or alkali- or alkaline earth hydroxides.

Preferred zwitterionic or amphoteric polymers are:

copolymers formed from alkylacrylamide, alkylaminoalkyl methacrylate, and two or more monomers from acrylic acid and methacrylic acid as well as, if necessary, their esters, especially copolymers from octylacrylamide, acrylic acid, butylaminoethyl methacrylate, methyl methacrylate and hydroxypropyl methacrylate (INCI designation: Octylacrylamide/Acrylate/Butylaminoethyl Methacrylate Copolymer); copolymers, that are formed from at least one of a first type of monomer that possesses quaternary amino groups and at least one of a second type of monomer that possesses acid groups; copolymers from fatty alcohol acrylates, alkylamine oxide methacrylate and at least one monomer selected from acrylic acid and methacrylic acid as well as if necessary acrylic acid esters and methacrylic acid esters, especially copolymers from lauryl acrylate, stearyl acrylate, ethylamine oxide methacrylate and at least one monomer selected from acrylic acid and methacrylic acid as well as if necessary their esters; copolymers from methacryloyl ethyl betaine and at least one monomer selected from methacrylic acid and methacrylic acid esters; copolymers from acrylic acid, methyl acrylate and methacrylamidopropyltrimethylammonium chloride (INCI designation: Polyquatemium-47); copolymers from acrylamidopropyltrimethylammonium chloride and acrylates or copolymers from acrylamide, acrylamidopropyltrimethylammonium chloride, 2-amidopropylacrylamide sulfonate, and dimethylaminopropylamine (INCI designation: Polyquaternium-43); oligomers or polymers, producible from quaternary crotonoylbetaines or quaternary crotonoylbetaine esters.

In one embodiment, the agent according to the present invention, as a hair-conditioning or hair-setting additive, contains at least one cationic polymer. The cationic polymers are contained in the composition to be used according to the present invention in a quantity that is preferably 0.01 to 20 wt. % or 0.05 to 1.0 wt. %, with 0.1 to 5 wt. % being particularly preferred. The polymers can be synthetic or natural polymers. The polymers are hair-setting and/or hair-conditioning polymers that preferably form a film as well. Natural polymers are understood to also include chemically modified polymers of natural origin. Hair-setting polymers are understood to be those capable of exhibiting a setting effect on the hair or a stabilizing effect on the hairstyle when used in a 0.01 to 5% aqueous, alcoholic, or aqueous alcoholic solution or dispersion, e.g., those that increase curl retention with respect to a water wave, especially those for which the “Hair Fixatives” function is indicated in the International Cosmetic Ingredient Dictionary and Handbook, 10th edition, 2004. Hair-conditioning polymers are understood to be those capable of exhibiting a hair-conditioning or conditioning effect on the hair when used in a 0.01 to 5% aqueous, alcoholic, or aqueous alcoholic solution or dispersion, e.g., those that improve the combing ability or increase shine, especially those for which the “Hair Conditioning Agents” finction is indicated in the International Cosmetic Ingredient Dictionary and Handbook, 10th edition, 2004. Film-forming polymers are understood to be those capable of depositing a polymer film on the hair after drying when used in a 0.01 to 5% aqueous, alcoholic, or aqueous alcoholic solution or dispersion, especially those for which the “Film Formers” function is indicated in the International Cosmetic Ingredient Dictionary and Handbook, 10th edition, 2004. The polymers can also simultaneously have two or three of the properties known as “film-forming,” “hair-setting,” and “hair-conditioning.”

Cationic polymers are polymers with cationic groups or with amine groups, particularly primary, secondary, tertiary, or quaternary amine groups. The cationic charge density will preferably be 1 to 7 meq/g.

Suitable synthetic cationic polymers are homo- or copolymers consisting of at least one of the following monomers:dialkylaminoalkyl acrylate, dialkylaminoalkyl methacrylate, monoalkylaminoalkyl acrylate, and monoalkyl aminoalkyl methacrylate, trialkyl methacryloxyalkyl ammonium, trialkyl acryloxyalkyl ammonium, dialkyl diallyl ammonium, and quaternary vinyl ammonium monomers with cyclic groups containing cationic nitrogens.

Suitable cationic polymers preferably contain quaternary amino groups. Cationic polymers can be homo- or copolymers, where the quaternary nitrogen groups are contained either in the polymer chain or preferably as substituents on one or more of the monomers. The monomers containing ammonium groups can be copolymerized with non-cationic monomers. Suitable cationic monomer are unsaturated compounds that can undergo radical polymerization, which bear at least one cationic group, especially ammonium-substituted vinyl monomers such as, for example, trialkylmethacryloxyalkylammonium, trialkylacryloxyalkylammonium, dialkyldiallyl ammonium and quaternary vinylammonium monomers with cyclic, cationic nitrogen-containing groups such as pyridinium, imidazolium or quaternary pyrrolidones, e.g., alkylvinylimidazolium, alkylvinylpyridinium, or alkylvinylpyrrolidone salts. The alkyl groups of these monomers are preferably lower alkyl groups such as, for example, C1 to C7 alkyl groups, and especially preferred are C1 to C3 alkyl groups.

The monomers containing ammonium groups can be copolymerized with non-cationic monomers. Suitable comonomers are, for example, acrylamide, methacrylamide, alkyl- and dialkylacrylamide, alkyl- and dialkylmethacrylamide, alkyl acrylate, alkyl methacrylate, vinylcaprolactone, vinylcaprolactam, vinylpyrrolidone, vinyl esters, for example vinyl acetate, vinyl alcohol, propylene glycol or ethylene glycol, wherein the alkyl groups of these monomers are preferably C1 to C7 alkyl groups, and especially preferred are C1 to C3 alkyl groups.

Suitable polymers with quaternary amino groups are, for example, those described in the CTFA Cosmetic Ingredient Dictionary under the designations Polyquaternium such as methylvinylimidazolium chloride/vinylpyrrolidone copolymer (Polyquaternium-16) or quaternized vinylpyrrolidone/dimethylaminoethyl methacrylate copolymer (Polyquaternium-11) as well as quaternary silicone polymers or silicone oligomers such as, for example, silicone polymers with quaternary end groups (Quaternium-80).

Preferred cationic polymers of synthetic origin:

poly(dimethyldially lammonium chloride); copolymers from acrylamide and dimethyldiallyl ammonium chloride; quaternary ammonium polymers, formed by the reaction of diethyl sulfate with a copolymer from vinylpyrrolidone and dimethylaminoethyl methacrylate, especially vinylpyrrolidone/dimethylaminoethyl methacrylate methosulfate copolymer (e.g., Gafquat® 755 N, Gafquat® 734); quaternary ammonium polymers from methylvinylimidazolium chloride and vinylpyrrolidone (e.g., LUVIQUAT® HM 550); Polyquatemium-35; Polyquaternium-57; polymers from trimethylammonium ethyl methacrylate chloride; terpolymers from dimethyldiallyl ammonium chloride, sodium acrylate and acrylamide (e.g., Merquat® Plus 3300); copolymers from vinylpyrrolidone, dimethylaminopropyl methacrylamide and methacryloylaminopropyllauryldimethylammonium chloride; terpolymers from vinylpyrrolidone, dimethylaminoethyl methacrylate and vinylcaprolactam (e.g., Gaffix® VC 713); vinylpyrrolidone/methacrylamidopropyltrimethylammonium chloride copolymers (e.g., Gafquat® HS 100); copolymers from vinylpyrrolidone and dimethylaminoethyl methacrylate; copolymers from vinylpyrrolidone, vinylcaprolactam and dimethylaminopropylacrylamide; poly- or oligoesters formed from at least one first type of monomer, that is selected from hydroxyacids substituted with at least one quaternary ammonium group; dimethylpolysiloxane substituted with quaternary ammonium groups in the terminal positions.

Suitable cationic polymers that are derived from natural polymers are especially cationic derivatives of polysaccharides, for example, cationic derivatives of cellulose, starch or guar. Furthermore, chitosan and chitosan derivatives are also suitable. Cationic polysaccharides are, for example, represented by the general formula
G-O—B—N⁺R^{a}R^{b}R^{c}X⁻

G is an anhydroglucose residue, for example, starch or cellulose anhydroglucose;

B is a divalent linking group, for example alkylene, oxyalkylene, polyoxyalkylene or hydroxyalkylene;

R^{a}, R^{b}, and R^{c}, independently from one another, are alkyl, aryl, alkylaryl, arylalkyl, alkoxyalkyl, or alkoxyaryl, any of which can have up to 18 C atoms, wherein the total number of C atoms in R^{a}, R^{b}, and R^{c} is preferably a maximum of 20;

X is a conventional counter-anion, for example, a halide, acetate, phosphate, nitrate, or alkyl sulfate, preferably a chloride. Cationic celluloses are, for example, those with the INCI names Polyquaternium4, Polyquaternium-10, or Polyquaternium-24. A suitable cationic guar derivative has, for example, the INCI designation Guar Hydroxypropyltrimonium Chloride.

Especially preferred cationically-active substances are chitosan, chitosan salts and chitosan derivatives. Chitosans that can be used according to the invention can be fully or partially deacetylated chitins. By way of example, the molecular weight can be distributed over a broad range, from 20,000 to about 5 million g/mol, for example from 30,000 to 70,000 g/mol. However, the molecular weight will preferably lie above 100,000 g/mol, and especially preferred from 200,000 to 700,000 g/mol. The degree of deacetylation is preferably from 10 to 99%, and especially preferably from 60 to 99%. A preferred chitosan salt is chitosonium pyrrolidone carboxylate, e.g., Kytamer® PC with a molecular weight of from about 200,000 to 300,000 g/mol and a degree of deacetylation of from 70 to 85%. Chitosan derivatives that can be considered include quaternized, alkylated or hydroxyalkylated derivatives, e.g., hydroxyethyl, hydroxypropyl or hydroxybutyl chitosan. The chitosans or chitosan derivatives are preferably present in their neutralized or partially neutralized form. The degree of neutralization will be preferably at least 50%, especially preferably between 70 and 100%, as calculated on the basis of the number of free base groups. For the neutralization agent, in principle any cosmetically compatible inorganic or organic acids can be used such as, for example, formic acid, tartaric acid, malic acid, lactic acid, citric acid, pyrrolidone carboxylic acid, hydrochloric acid and others, of which pyrrolidone carboxylic acid is especially preferred.

Preferred cationic polymers derived from natural sources:

cationic cellulose derivatives from hydroxyethyl cellulose and diallyldimethyl ammonium chloride; cationic cellulose deviates from hydroxyethyl cellulose and trimethylammonium-substituted epoxide; chitosan and its salts; hydroxyalkyl chitosans and their salts; alkylhydroxyalkyl chitosans and their salts; N-hydroxyalkylchitosan alkyl ethers.

In another preferred embodiment, the agent according to the present invention contains 0.01 to 15 wt. %, or preferably 0.5 to 10 wt. %, of at least one synthetic or natural nonionic film-forming polymer. Natural polymers are understood to also include chemically modified polymers of natural origin. Film-forming polymers are understood to be polymers that, when applied in a 0.01 to 5% aqueous, alcoholic, or aqueous-alcoholic solution, are capable of depositing a polymer film on the hair. Suitable synthetic nonionic polymers are homo- or copolymers consisting of at least one of the following monomers: vinyl lactams such as, for example, vinyl pyrrolidone or vinyl caprolactam; vinyl esters such as, for example, vinyl acetate; vinyl alcohol, vinyl formamide, acrylamides, methacrylamides, alkyl acrylamides, dialkylacrylamides, alkyl methacrylamides, dialkylmethacrylamides, alkyl acrylates, alkyl methacrylates, alkyl maleimides such as, for example, ethylmaleimide or hydroxyethylmaleimide, and alkylene glycols such as, for example, propylene glycol or ethylene glycol, wherein the alkyl and/or alkylene groups of these monomers are preferably C1 to C7 alkyl groups, with C1 to C3 alkyl groups being particularly preferred.

Suitable homopolymers are, for example, those of vinylcaprolactam, vinylpyrrolidone or N-vinylformamide. Further suitable synthetic, nonionic polymers are, for example, polyacrylamides, polyethylene glycol/polypropylene glycol copolymers, copolymerides from vinylpyrrolidone and vinyl acetate, terpolymers from vinylpyrrolidone, vinyl acetate, and vinyl propionate, polyacrylamides; polyvinyl alcohols as well as polyethylene glycol/polypropylene glycol copolymers. Suitable natural film-forming polymers are, in particular, those based on saccharide, preferably glucans, e.g., cellulose and derivatives thereof. Suitable derivatives are, in particular, those with alkyl and/or hydroxyalkyl substituents, wherein the alkyl groups can have, for example, 1 to 20, or preferably 1 to 4 C atoms, e.g., hydroxyalkyl cellulose. Preferred nonionic polymers are: polyvinylpyrrolidone, polyvinylcaprolactam, vinylpyrrolidone/vinyl acetate copolymers, polyvinyl alcohol, isobutylene/ethylmaleimide/hydroxyethylmaleimide copolymer; copolymers from vinylpyrrolidone, vinyl acetate, and vinyl propionate.

In one embodiment, the agent according to the invention contains, as a hair-conditioning active ingredient, at least one silicone compound preferably in a quantity of 0.01 to 15 wt. %, with 0.1 to 5 wt. % being especially preferred. The silicone compounds include volatile and nonvolatile silicones and silicones that are soluble and insoluble in the agent. One embodiment is high-molecular-weight silicone with a viscosity of 1,000 to 2,000,000 cSt at 77° F. (25° C.), or preferably 10,000 to 1,800,000 or 100,000 to 1,500,000. The silicone compounds include polyalkyl and polyaryl siloxanes, particularly with methyl, ethyl, propyl, phenyl, methylphenyl, and phenylmethyl groups. Polydimethyl siloxanes, polydiethyl siloxanes, and polymethylphenyl siloxanes are preferred. Also preferred are shine-providing, arylated silicones with a refractive index of at least 1.46 or at least 1.52. The silicone compounds include, in particular, the materials with the INCI designations Cyclomethicone, Dimethicone, Dimethiconol, Dimethicone Copolyol, Phenyl Trimethicone, Amodimethicone, Trimethylsilylamodimethicone, Stearyl Siloxysilicate, Polymethylsilsesquioxane, and Dimethicone Crosspolymer. Silicone resins and silicone elastomers are also suitable, wherein these are highly crosslinked siloxanes. Crosslinked silicones can be used simultaneously to provide consistency to the preferably creamy, solid, or highly viscous composition. Crosslinked silicones are, for example, those with the INCI designations Acrylates/Bis-Hydroxypropyl Dimethicone Crosspolymer, Butyl Dimethiconemethacrylate/Methyl Methacrylate Crosspolymer, C30-45 Alkyl Cetearyl Dimethicone Crosspolymer, C30-45 Alkyl Dimethicone/ Polycyclohexene Oxide Crosspolymer, Cetearyl Dimethicone/Vinyl Dimethicone Crosspolymer, Dimethicone Crosspolymer, Dimethicone Crosspolymer-2, Dimethicone Crosspolymer-3, Dimethicone/Divinyldimethicone/Silsesquioxane Crosspolymer, Dimethicone/PEG-10/15 Crosspolymer, Dimethicone/PEG-15 Crosspolymer, Dimethicone/PEG-10 Crosspolymer, Dimethicone/Phenyl Vinyl Dimethicone Crosspolymer, Dimethicone/Polyglycerin-3 Crosspolymer, Dimethicone/Titanate Crosspolymer, Dimethicone/Vinyl Dimethicone Crosspolymer, Dimethicone/Vinyltrimethylsiloxysilicate Crosspolymer, Dimethiconol/Methylsilanol/Silicate Crosspolymer, Diphenyl Dimethicone Crosspolymer, Diphenyl Dimethicone/Vinyl Diphenyl Dimethicone/Silsesquioxane Crosspolymer, Divinyldimethicone/Dimethicone Crosspolymer, Lauryl Dimethicone PEG-15 Crosspolymer, Lauryl Dimethicone/Polyglycerin-3 Crosspolymer, Methylsilanol/Silicate Crosspolymer, PEG-10 Dimethicone Crosspolymer, PEG-12 Dimethicone Crosspolymer, PEG-10 Dimethicone/Vinyl Dimethicone Crosspolymer, PEG-10/Lauryl Dimethicone Crosspolymer, PEG-15/Lauryl Dimethicone Crosspolymer, Silicone Quatemium-16/Glycidoxy Dimethicone Crosspolymer, Styrene/Acrylates/Dimethicone Acrylate Crosspolymer, Trifluoropropyl Dimethicone/PEG-10 Dimethicone Crosspolymer, Trifluoropropyl Dimethicone/Trifluoropropyl Divinyldimethicone Crosspolymer, Trifluoropropyl Dimethicone/Vinyl Trifluoropropyl Dimethicone/Silsesquioxane Crosspolymer, Trimethylsiloxysilicate/Dimethicone Crosspolymer, Trimethylsiloxysilicate/Dimethiconol Crosspolymer, Vinyl Dimethicone/Lauryl Dimethicone Crosspolymer, Vinyl Dimethicone/Methicone Silsesquioxane Crosspolymer, and Vinyldimethyl/Trimethylsiloxysilicate Stearyl Dimethicone Crosspolymer.

Preferred silicones are: cyclic dimethyl siloxanes, linear polydimethyl siloxanes, block polymers from polydimethyl siloxane and polyethylene oxide and/or polypropylene oxide, polydimethyl siloxanes with terminal or lateral polyethylene oxide or polypropylenoxide radicals, polydimethyl siloxanes with terminal hydroxyl groups, phenyl-substituted polydimethyl siloxanes, silicone emulsions, silicone elastomers, silicone waxes, silicone gums, amino-substituted silicones, silicones substituted with quaternary ammonia groups, and crosslinked silicones.

In one embodiment, the agent according to the present invention contains a photoprotective material preferably in a quantity of from 0.01 to 10 wt. % or of from 0.1 to 5 wt. %, with 0.2 to 2 wt. % being especially preferred. The photoprotective materials include, in particular, all the photoprotective materials mentioned in EP 1 084 696. The following are preferred: 4-methoxy cinnamic acid-2-ethylhexyl ester, methyl methoxy cinnamate, 2-hydroxy-4-methoxy benzophenone-5-sulfonic acid, and polyethoxylated p-aminobenzoate.

In one embodiment, the agent according to the present invention contains 0.01 to 20 wt. %, especially preferably 0.05 to 10 wt. %, or very especially preferably 0.1 to 5 wt. % of at least one hair-conditioning additive, selected from betaine; panthenol; panthenyl ethyl ether; sorbitol; protein hydrolysates; plant extracts; A-B block copolymers from alkyl acrylates and alkyl methacrylates; A-B block copolymers from alkyl methacrylates, and acrylonitrile; A-B-A block copolymers from lactide and ethylene oxide; A-B-A block copolymers from caprolacton and ethylene oxide; A-B-C block copolymers from alkylene or alkadiene compounds, styrene and alkyl methacrylates; A-B-C block copolymers from acrylic acid, styrene, and alkyl methacrylates; star-shaped block copolymers; hyper-branched polymers; dendrimers; intrinsically electrically conducting 3,4-polyethylene dioxythiophenes and intrinsically electrically conducting polyanilines.

In one embodiment, the agent according to the invention contains 0.01 to 5 wt. %, or especially preferably 0.05 to 1 wt. %, of at least one preservative. Suitable preservatives are those materials listed with the “Preservatives” function in the International Cosmetic Ingredient Dictionary and Handbook, 10th edition, e.g., phenoxyethanol, benzylparaben, butylparaben, ethylparaben, isobutylparaben, isopropylparaben, methylparaben, propylparaben, iodopropynyl butylcarbamate, methyldibromoglutaronitrile, and DMDM hydantoin.

A particular embodiment of the invention relates to a hair-conditioning agent. Hair-conditioning agents are, for example, conditioners, treatments, hair-repair products, rinses, and the like. The hair-conditioning agent contains at least one hair-conditioning ingredient selected from the aforementioned silicone compounds, cationic or amine-substituted surfactants, and cationic or amine-substituted polymers. The hair-conditioning agent can be used in quantities of between 0.01 and 10.0 wt. %, or particularly between 0.01 and 5.0 wt. %, based on the finished product. The hair-conditioning agent according to the invention can, after application to the dry, damp, or wet hair, either remain in the hair or it can be rinsed out after a suitable action period. The action times depend on the type of hair. As a general rule, action times of between 0.5 and 30 minutes, or particularly 0.5 and 10 minutes, or preferably between 1 and 5 minutes can be assumed.

In addition to the aforementioned cationic surfactants, other suitable cationic or amino-substituted surfactants are those of the formula R1-NH—(CH₂)n—NR2R3

or of the formula R1-NH—(CH₂)n—N⁺R2R3R4X⁻

wherein R1 is an acyl or an alkyl residue with 8 to 24 C atoms, which can be branched or linear, saturated or unsaturated, whereby the acyl and/or the alkyl residue can contain one or more OH groups, R2, R3 and R4 independently of one another are hydrogen, alkyl or alkoxyalkyl residues with 1 to 6 C atoms, which can be the same or different, saturated or unsaturated and can be substituted with one or more hydroxy groups, X— is an anion, especially a halide ion or a compound of the general formula RSO₃—, wherein R has the meaning of saturated or unsaturated alkyl residues with 1 to 4 C atoms, and n means a whole number between 1 and 10, preferably from 2 to 5.

The active hair-conditioning compound is preferably an amidoamine and/or a quaternized amidoamine of the aforementioned formula, wherein R1 is a branched or linear, saturated or unsaturated acyl residue with 8 to 24 C atoms that can contain at least one OH group. Preferred are such amines and/or quaternized amines, in which at least one of the residues R2, R3 and R4 means a residue according to the general formula CH₂CH₂OR5, wherein R5 can have the meaning of alkyl residues with 1 to 4 C atoms, hydroxyethyl or H. Suitable amines or amidoamines, which can be optionally quaternized, are especially such with the INCI names Ricinoleamidopropyl Betaine, Ricinoleamidopropyl Dimethylamine, Ricinoleamidopropyl Dimethyl Lactate, Ricinoleamidopropyl Ethyldimonium Ethosulfate, Ricinoleamidopropyltrimonium Chloride, Ricinoleamidopropyltrimonium Methosulfate, Cocamidopropyl Betaine, Cocamidopropyl Dimethylamine, Cocamidopropyl Ethyldimonium Ethosulfate, Cocamidopropyltrimonium Chloride, Behenamidopropyl Dimethylamine, Isostearylamidopropyl Dimethylamine, Stearylamidopropyl Dimethylamine, Quaternium-33, Undecyleneamidopropyltrimonium Methosulfate.

In a preferred embodiment, the agent according to the invention contains at least one pigment. The pigments can be colored pigments that provide coloring effects to the product mass or the hair, or they can be shine-enhancing pigments that provide shine effects to the product or the hair. The color or shine effects in the hair are preferably temporary, i.e., they remain until the next time the hair is washed and can be removed by washing the hair with typical shampoos. The pigments are not dissolved in the product mass and can be contained in a quantity of from 0.01 to 25 wt. %, with 5 to 15 wt. % being particularly preferred. The preferred particle size is 4×10⁻⁵-0.008 in (1 to 200 μm), or particularly 0.0001-0.006 in (3 to 150 μm), and especially preferably 0.0004-0.004 in (10 to 100 μm). The pigments are practically insoluble colorants in the application medium and can be inorganic or organic. Inorganic-organic mixed pigments are also possible. Inorganic pigments are preferred. The advantage of inorganic pigments is their extraordinary resistance to light, weather, and temperature. The inorganic pigments can be of natural origin, for example, manufactured from chalk, ocher, umbra, green earth, burnt Terra di Siena, or graphite. The pigments can also be white pigments such as, for example, titanium dioxide or zinc oxide; black pigments such as, for example, iron oxide black; color pigments such as, for example, ultramarine or iron oxide red; shine pigments; metal effect pigments; pearl shine pigments; as well as fluorescence or phosphorescence pigments; wherein it is preferred if at least one pigment is a colored, nonwhite pigment. Metallic oxides, metallic hydroxides, and metallic oxide hydrates, mixed phase pigments, sulfur-containing silicates, metallic sulfides, complex metal cyanides, metallic sulfates, metallic chromates, and metallic molybdates, as well as the metals themselves (bronze pigments) are suitable. Titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (CI77289), iron blue (ferric ferrocyanide, C177510), and carmine (cochineal) are particularly suitable.

Pearl-shine and color pigments based on mica and/or glimmer that are coated with a metallic oxide or a metallic oxychloride such as titanium dioxide or bismuth oxychloride as well as, if necessary, other color-providing materials such as iron oxides, iron blue, ultramarine, carmine, etc., and wherein the color can be determined by varying the thickness of the coat, are especially preferred. These types of pigments are sold, for example, under the trade names Rona®, Colorona®, Dichrona®, and Timiron® by Merck, in Germany.

Organic pigments are, for example, the natural pigments sepia, Garcinia gummi-gutta, bone black, Van Dyke brown, indigo, chlorophyll, and other plant pigments. Synthetic organic pigments are, for example, azo-pigments, anthraquinoids, indigoids, and dioxazine, quinacridone, phthalocyanine, isoindolinone, perylene, perinone, metallic complex, alkali blue, and diketopyrrolopyrrol pigments.

In one embodiment, the agent according to the present invention contains 0.01 to 10 wt. %, or especially preferably 0.05 to 5 wt. %, of at least one particle-shaped material. Suitable materials are, for example, materials that are solid and in the form of particles at room temperature (77° F. (25° C.)). Silica, silicates, aluminates, alumina, mica, salts, particularly inorganic metallic salts, metallic oxides, e.g., titanium dioxide, minerals, and polymer particles are somewhat suitable. The particles are present in the agent in an undissolved, preferably steadily dispersed form and can be deposited on the hair in solid form after being applied to the hair and after the solvent has evaporated. A stable dispersion can be obtained by providing the composition with a yield point that is great enough to inhibit any sinking of the solid particles. A sufficient yield point can be obtained by using suitable gel-formers in a suitable quantity. Preferred particle-shaped materials are silica (silica gel, silicium dioxide) and metallic salts, particularly inorganic metallic salts, wherein silica is especially preferred. Metallic salts are, for example, alkaline or alkaline-earth halogenides such as sodium chloride or potassium chloride; and alkaline or alkaline earth sulfates such as sodium sulfate or magnesium sulfate.

An additional embodiment relates to an agent for permanently restructuring hair. It contains at least one reducing agent, particularly a keratin-reducing mercapto compound preferably in a quantity of from 0.5 to 15 wt. %. The permanent wave agent is preferably adjusted to be an aqueous, alkaline (pH=5 to 10) preparation, which contains, for example, cysteine, cysteamine, N-acetyl-L-cysteine, mercapto carboxylic acids, such as, for example, mercaptoacetic acid or thiolactic acid, or salts of mercapto carboxylic acids, such as, for example, ammonium and guanidine salts of mercaptoacetic acid or thiolactic acid as a keratin-reducing mercapto compound. The required alkalinity is obtained by adding ammonia, organic amines, ammonium and alkali carbonates, or bicarbonates. Neutral or acidic (pH=4.5 to 7) hair restructuring agents that have an effective content of sulfites or mercaptocarboxylic acid esters in an aqueous medium can also be considered. In the first case, preferably sodium or ammonium sulfite or the salt of sulfuric acid with an organic amine such as, for example, monoethanolamine and guanidine, can be used in a concentration of approximately 2 to 12 wt. % (calculated as SO2). In the latter case, mercaptoacetic acid mono glycol esters or glycerin esters are particularly used in a concentration of approximately 5 to 50 wt. % (corresponding to a content of 2 to 16 wt. % mercaptoacetic acid). The agent according to the invention for permanent restructuring of hair can also contain a mixture of the aforementioned keratin-reducing compounds. For the oxidative after-treatment, a fixing agent according to the invention containing at least one oxidizing agent can be used. Examples of oxidizing agents that can be used in one of these types of fixing agents are sodium and potassium bromate, sodium perborate, urea peroxide, and hydrogen peroxide. The concentration of oxidizing agent can be approximately 0.5 to 10 wt. %. Both the agent according to the invention for permanent hair restructuring as well as the fixing agent according to the invention can be present in the form of an emulsion or in thickened form on an aqueous basis, particularly as a cream, gel, or paste.

The composition to be used according to the invention can further contain any additive components that are conventional for hair treatment agents, for example perfume oils; opacifying agents such as, for example, ethylene glycol distearate, styrene/PVP copolymers or polystyrenes; humectants; shine providers; product dyes; antioxidants; each preferably in quantities of 0.01 to 10 wt. %, wherein the total quantity preferably does not exceed 10 wt. %.

The object of the invention is also a method for hair treatment, wherein
a product release system according to the invention is provided,
via the product release system, the composition contained therein is sprayed on the hair, and
the composition that is sprayed on is either rinsed out of the hair after an action period or it is left in the hair.

Instead of being sprayed directly onto the hair, the product can also be placed in the hands or on an application device such as, for example, a comb or a brush, and then distributed into the hair, particularly if the product has a snow-like consistency, or it is in the form of flakes or foam.

The products according to the invention are characterized, constrained by their special application with the special aerosol spray system to be used according to the invention, by an excellent distribution capacity in conjunction with a good hairstyle stability with good hold as well as shine for the hair. The advantages with the application are shown in the comfortable application, the more economical dispensing, the consistency that is perceived by the user as being more pleasant, and the more pleasant feel on the skin. An additional advantage of the products according to the present invention is that differing spray properties can be precisely adjusted by simply varying the propellant, the propellant composition, or the propellant pressure; these spray properties were not previously possible for the underlying active ingredient compositions. The spray properties include everything from a fine aerosol atomized spray and snow-like drops to flakes of spray and spray foam.

The following examples should serve to illustrate further the object of the present invention.

### EXAMPLES

In the following examples, the individual active ingredient compositions were filled, along with the individually indicated propellants, into a pressure-resistant aerosol can and equipped with a capillary spray system, as can be obtained, for example, under the trade name TRUSPRAY® from Boehringer Ingelheim microParts GmbH.

### Example 1

### Hair Styling Gel

Active ingredient composition:

| Raw ingredients | Quantity | |
|---|---|---|
| Luviset ® Clear ¹⁾ | 0.04 | oz (1.00 g) |
| VA/Crotonates copolymer | 0.09 | oz (2.50 g) |
| Acrylates/ceteth-20-itaconate copolymer | 0.02 | oz (0.50 g) |
| Sorbitol | 0.15 | oz (4.20 g) |
| Carbomer | 0.04 | oz (1.20 g) |
| Aminomethyl propanol, 95% | 0.01 | oz (0.30 g) |
| Methylparaben | 0.007 | oz (0.20 g) |
| PEG-40 hydrogenated castor oil | 0.007 | oz (0.20 g) |
| Panthenol | 0.004 | oz (0.10 g) |
| Perfume | 0.007 | oz (0.20 g) |
| Ethanol | 0.18 | oz (5.00 g) |
| Water | balance to 3.5 | oz (100) |

¹⁾ Terpolymer from vinyl pyrrolidone, methacrylamide, and vinylimidazole (BASF)
Consistency: Highly viscous, clear gel

Filling with propellant:

| | 1-1 | 1-2 | 1-3 | 1-4 | 1-5 |
|---|---|---|---|---|---|
| Active ingredient | 1.8 oz | 1.8 oz | 1.8 oz | 2.3 oz | 1.4 oz |
| composition | (50 g) | (50 g) | (50 g) | (65 g) | (40 g) |
| Propane/butane 4.8 bar | 1.8 oz | | | | |
| | (50 g) | | | | |
| Propane/butane 2.7 bar | | 1.8 oz | | | |
| | | (50 g) | | | |
| DME | | | 1.8 oz | 1.2 oz | 2.1 oz |
| | | | (50 g) | (35 g) | (60 g) |

Spray properties:

| | |
|---|---|
| 1-1: | Snow-like spray |
| 1-2: | Snow-like spray |
| 1-3: | Wet aerosol spray |
| 1-4: | Spray foam |
| 1-5: | Droplets (snow-like) |

### Example 2

### Hair Styling Cream

Composition:

| Raw ingredients | Quantity | |
|---|---|---|
| Carbomer | 0.04 | oz (1.00 g) |
| AMP | 0.03 | oz (0.90 g) |
| Guar gum | 0.01 | oz (0.30 g) |
| Polyvinylpyrrolidone | 0.11 | oz (3.00 g) |
| Dimethicone | 0.529 | oz (15.00 g) |
| PEG-40 hydrogenated castor oil | 0.007 | oz (0.20 g) |
| Perfume | 0.007 | oz (0.20 g) |
| Ethanol | 0.881 | oz (25.00 g) |
| Water | balance to 3.5 | oz (100 g) |

Consistency: Highly viscous cream

Filling with propellant:

| | 2-1 | 2-2 | 2-3 | 2-4 | 2-5 |
|---|---|---|---|---|---|
| Active ingredient | 1.8 oz | 1.8 oz | 1.8 oz | 2.1 oz | 2.5 oz |
| composition | (50 g) | (50 g) | (50 g) | (60 g) | (70 g) |
| Propane/butane 4.8 bar | 1.8 oz | | | 1.4 oz | 1.1 oz |
| | (50 g) | | | (40 g) | (30 g) |
| Propane/butane 2.7 bar | | 1.8 oz | | | |
| | | (50 g) | | | |
| DME | | | 1.8 oz | | |
| | | | (50 g) | | |

Spray properties:

| | |
|---|---|
| 2-1: | Fine aerosol spray |
| 2-2: | Wet aerosol spray |
| 2-3: | Spray foam |
| 2-4: | Fine aerosol spray |
| 2-5: | Fine aerosol spray |

### Example 3

### Hair Styling Wax

Composition:

| Raw ingredients | Quantity | |
|---|---|---|
| Low viscosity paraffin | 1.058 | oz (30.00 g) |
| Ceresin | 0.60 | oz (17.00 g) |
| TRICETEARETH-4 PHOSPHATE | 0.35 | oz (10.00 g) |
| PEG-25 HYDROGENATED | 0.09 | oz (2.50 g) |
| CASTOR OIL | | |
| Carnauba wax | 0.18 | oz (5.00 g) |
| Beeswax | 0.28 | oz (8.00 g) |
| Propylparabens | 0.007 | oz (0.20 g) |
| Perfume | 0.01 | oz (0.30 g) |
| Glyceryl polyacrylate | 0.19 | oz (5.50 g) |
| Methylparaben | 0.01 | oz (0.30 g) |
| Ethanol | 0.30 | oz (8.50 g) |
| Water | balance to 3.5 | oz (100 g) |

Consistency: Very pasty wax

Filling with propellant:

| | 3-1 | 3-2 | 3-3 | 3-4 | 3-5 |
|---|---|---|---|---|---|
| Active ingredient | 1.8 oz | 1.8 oz | 1.8 oz | 2.5 oz | 2.1 oz |
| composition | (50 g) | (50 g) | (50 g) | (70 g) | (60 g) |
| Propane/butane 4.8 bar | 1.8 oz | | | 1.1 oz | 1.4 oz |
| | (50 g) | | | (30 g) | (40 g) |
| Propane/butane 2.7 bar | | 1.8 oz | | | |
| | | (50 g) | | | |
| DME | | | 1.8 oz | | |
| | | | (50 g) | | |

### Example 4

### Microemulsion

| Raw ingredients | Quantity | |
|---|---|---|
| Cetearyl alcohol | 0.35 | oz (10.00 g) |
| Low viscosity paraffin | 0.14 | oz (4.00 g) |
| Isopropyl myristate | 0.14 | oz (4.00 g) |
| Acrylates/lauryl acrylate/ | 0.05 | oz (1.50 g) |
| stearyl acrylate/ethylamine oxide | | |
| methacrylate copolymer | | |
| Steareth-20 | 0.05 | oz (1.50 g) |
| Carbomer | 0.01 | oz (0.30 g) |
| Aminomethylpropanol | 0.002 | oz (0.05 g) |
| Perfume | 0.014 | oz (0.40 g) |
| Methylparaben | 0.01 | oz (0.30 g) |
| Water | balance to 3.5 | oz (100 g) |

Consistency: Thick cream

Propellant fillers:

| | 4-1 | 4-2 | 4-3 | 4-4 | 4-5 |
|---|---|---|---|---|---|
| Active ingredient | 1.8 oz | 1.8 oz | 1.8 oz | 2.1 oz | 2.5 oz |
| composition | (50 g) | (50 g) | (50 g) | (60 g) | (70 g) |
| Propane/butane 4.8 bar | 1.8 oz | | | | |
| | (50 g) | | | | |
| Propane/butane 2.7 bar | | 1.8 oz | | | |
| | | (50 g) | | | |
| DME | | | 1.8 oz | 1.4 oz | 1.1 oz |
| | | | (50 g) | (40 g) | (30 g) |

Spray properties:

| | |
|---|---|
| 4-1: | Snow-like spray |
| 4-2: | Snow-like spray |
| 4-3: | Wet aerosol spray |
| 4-4: | Spray foam |
| 4-5: | Spray foam |

### Example 5

### Solid Microemulsion

Active ingredient composition:

| Raw ingredients | Quantity | |
|---|---|---|
| Low viscosity paraffin | 0.49 | oz (13.8 g) |
| Oleth-10 | 0.44 | oz (12.5 g) |
| OLETH-5 | 0.44 | oz (12.5 g) |
| Polyquaternium-22 | 0.09 | oz (2.5 g) |
| PEG-40 hydrogenated castor oil | 0.07 | oz (2.0 g) |
| Perfume | 0.007 | oz (0.2 g) |
| Dekaben LMB | 0.007 | oz (0.2 g) |
| Water | balance to 3.5 | oz (100 g) |

Consistency: Translucent, solid microemulsion

Filling with propellant:

| | 5-1 | 5-2 | 5-3 | 5-4 | 5-5 |
|---|---|---|---|---|---|
| Active ingredient | 1.8 oz | 2.1 oz | 2.5 oz | 2.8 oz | 1.4 oz |
| composition | (50 g) | (60 g) | (70 g) | (80 g) | (40 g) |
| Propane/butane 4.8 bar | 1.8 oz | | 1.1 oz | 0.35 oz | 2.1 oz |
| | (50 g) | | (30 g) | (10 g) | (60 g) |
| Propane/butane 2.7 bar | | 1.4 oz | | | |
| | | (40 g) | | | |
| DME | | | | 0.35 oz | |
| | | | | (10 g) | |

Spray properties:

| | |
|---|---|
| 5-1: | Fine, dry aerosol spray |
| 5-2: | Fine, wet aerosol spray |
| 5-3: | Snow-like droplet formation |
| 5-4: | Spray foam |
| 5-5: | Very fine, dry aerosol |

Practical tests were conducted on a mannequin wig. In a half-side comparison, the left side of the mannequin wig was treated with aerosol application 5-1 and the right side was treated with the propellant-free, highly viscous active ingredient composition. The aerosol version was easy to dispense and could be applied very precisely; it was also easier to distribute in the hair than the pure active ingredient. The hair treated with the aerosol application exhibited a significantly improved shine and significantly improved hold with the hairstyle in comparison to the hair treated with the pure active ingredient composition.

### Example 6

### Skin/Scalp Cleaning and Peeling Paste

| Sodium lauryl ether sulfate | 0.43 | oz (12.3 g) |
|---|---|---|
| Propylene glycol | 0.05 | oz (1.5 g) |
| Cocamidopropyl betaine | 0.05 | oz (1.5 g) |
| Acrylates/C10-30 alkyl acrylate | 0.04 | oz (1 g) |
| crosspolymer | | |
| PEG-3 distearate | 0.04 | oz (1 g) |
| Magnesium lauryl ether sulfate | 0.03 | oz (0.9 g) |
| Sodium oleyl ether sulfate | 0.014 | oz (0.4 g) |
| Magnesium oleyl ether sulfate | 0.004 | oz (0.1 g) |
| Zinc pyrithione | 0.04 | oz (1 g) |
| Climbazole | 0.02 | oz (0.6 g) |
| Piroctone olamine | 0.02 | oz (0.5 g) |
| Polyquaternium-10 | 0.004 | oz (0.1 g) |
| Preservative, perfume | q.s. | |
| Water | balance to 3.5 | oz (100 g) |

Viscosity (12.9 s⁻¹, 77° F. (25° C.)): 0.0008 lb/in² s (5,408 mPa s)

Fill ratios in wt. %:

| Example | Active ingredient solution | propane/butane 4.8 bar | DME |
|---|---|---|---|
| 6-A | 60 | 40 | |
| 6-B | 60 | | 40 |

When the filling is done with DME, there is a fine atomized spray. The atomized products foam up well on the scalp or hair together with water. When the filling is done with propane/butane, there is a snow-like spray comparable to artificial snow. The small white flakes foam well when combined with water. Product 6-B containing anti-dandruff agents is particularly advantageous, because it can be sprayed directly at the hair roots in the form of a fine atomized spray despite the pasty, highly viscous starting consistency.

### Example 7

### Shaving Cream

| | | |
|---|---|---|
| Stearic acid | 0.78 | oz (22 g) |
| Myristic acid | 0.49 | oz (14 g) |
| Glycerol | 0.42 | oz (12 g) |
| Potassium hydroxide | 0.28 | oz (8 g) |
| Coco fatty acids (INCI: Coconut Acid) | 0.24 | oz (6.8 g) |
| Triethanolamine | 0.06 | oz (1.8 g) |
| Potassium aluminum sulfate | 0.02 | oz (0.5 g) |
| (INCI: Potassium Alum) | | |
| Sodium hydroxide | 0.14 | oz (0.4 g) |
| Perfume | 0.14 | oz (0.4 g) |
| Disodium EDTA | 0.01 | oz (0.3 g) |
| Allantoin | 0.007 | oz (0.2 g) |
| Water | balance to 3.5 | oz (100 g) |

pH 8.0-9.0
Consistency: White, pasty cream

Fill ratios in wt. %:

| Example | Active ingredient solution | propane/butane 4.8 bar | DME |
|---|---|---|---|
| 7-A | 60 | 40 | |
| 7-B | 60 | | 40 |

When the filling is done with DME, there is a fine atomized spray during atomization. When the filling is done with propane/butane, flakes form during atomization.

### Example 8

### Hair Smoothing Gel

Active ingredient composition:

| | | |
|---|---|---|
| Ammonium thioglycolate | 0.64 | oz (18.2 g) |
| (70% aqueous solution) | | |
| Ammonia (25% aqueous solution) | 0.08 | oz (2.20 g) |
| Ammonium hydrogen carbonate | 0.12 | oz (3.50 g) |
| Polydimethyldiallyl ammonium | 0.03 | oz (0.9 g) |
| chloride | | |
| (INCI: Polyquaternium-6) | | |
| 1,2-propylene glycol | 0.09 | oz (2.50 g) |
| Glycerol | 0.11 | oz (3.00 g) |
| Cremophor ® EL (INCI: PEG-35 | 0.05 | oz (1.50 g) |
| Castor Oil) | | |
| Hydrogenated castor triglyceride | 0.04 | oz (1.00 g) |
| polyglycol ether | | |
| (INCI: PEG-40 Hydrogenated | | |
| Castor Oil) | | |
| Natrosol ® 250 HHX (INCI: | 0.05 | oz (1.5 g) |
| Hydroxyethyl Cellulose | | |
| Perfume oil | 0.02 | oz (0.5 g) |
| Water | balance to 3.5 | oz (100 g) |

pH 9.0; viscosity: 0.0007 lb/in² s (5100 mPas) (Haake Viskometer VT 550, SV DIN, shear speed 12.9 s⁻¹, 77° F. (25°C.))

The common propellants can be used; dimethylether is preferably used. The ingredient composition is filled in a weight ratio of ingredient : propellant =50:50 as well as in a weight ratio of 60:40.

Hair Treatment:

Spraying the gel on dry hair with the product release system according to the present invention and smoothing the hair with a comb (the comb can also be a part of the applicator), rinsing out after an action period (10 to 30 min), then setting (action period of the fixing agent is 5 to 12 min); then, if necessary, use of a hot smoothing iron.

Results: Smooth hair with excellent hold and very good combing properties; the product release system according to the invention enables the application of very viscous gel that doesn't drip off or run onto the scalp; the convenience and safety for the user is therefore significantly improved; user-friendly application of the thickened permanent wave agent; time-consuming application with the brush no longer necessary.

### Example 9

### Emulsion-Type Hair Cream

Composition:

| | A | | B | |
|---|---|---|---|---|
| Glyceryl monooleate | 1.1 | oz (32 g) | 1.1 | oz (32 g) |
| CAPRYLIC/CAPRIC TRIGLYCERIDE | 0.4 | oz (12 g) | 0.4 | oz (12 g) |
| Polyvinylpyrrolidone K30 | 0.18 | oz (5.23 g) | — | |
| Octylacrylamide/acrylate/butylaminoethyl | — | | 5 | g (0.2 oz) |
| methacrylate copolymer (Amphomer ®), | | | | |
| neutralized with aminomethyl propanol | | | | |
| PEG-60 HYDROGENATED CASTOR OIL | 0.2 | oz (5 g) | 0.2 | oz (5 g) |
| 1,2-propylene glycol | 0.13 | oz (3.6 g) | 0.13 | oz (3.6 g) |
| PEG/PPG-18/18 DIMETHICONE | 0.04 | oz (1 g) | 0.04 | oz (1 g) |
| PEG-12 DIMETHICONE | 0.04 | oz (1 g) | 0.04 | oz (1 g) |
| Cholesterol (fine cryst.) | 0.04 | oz (1 g) | 0.04 | oz (1 g) |
| OLETH-5 | 0.04 | oz (1 g) | 0.04 | oz (1 g) |
| Silicone oil (dimethylpolysiloxane, 20 mPa s) | 0.004 | oz (0.1 g) | 0.004 | oz (0.1 g) |
| | 0.1 | g (0.004 oz) | 0.1 | g (0.004 oz) |
| Perfume | 0.01 | oz (0.3 g) | 0.01 | oz (0.3 g) |
| Water | balance to 3.5 | oz (100 g) | balance to 3.5 | oz (100 g) |

Propellant fillers:

| | 9-1 | 9-2 | 9-3 | 9-4 | 9-5 |
|---|---|---|---|---|---|
| Active ingredient | 2.1 oz | 2.1 oz | 2.5 oz | 1.8 oz | 2.5 oz |
| | (60 g) | (60 g) | (70 g) | (50 g) | (70 g) |
| Propane/butane 4.8 bar | 1.4 oz | | 1.1 oz | 1.8 oz | |
| | (40 g) | | (30 g) | (50 g) | |
| Propane/butane 2.7 bar | | | | | 1.1 oz |
| | | | | | (30 g) |
| DME | | 1.4 oz | | | |
| | | (40 g) | | | |

| | 9-6 | 9-7 | 9-8 | 9-9 |
|---|---|---|---|---|
| Active ingredient | 2.1 oz | 1.8 oz | 1.9 oz | 1.8 oz |
| | (60 g) | (50 g) | (55 g) | (50 g) |
| Propane/butane 4.8 bar | | | | |
| Propane/butane 2.7 bar | 1.4 oz | 1.8 oz | | |
| | (40 g) | (50 g) | | |
| DME | | | 1.6 oz | 1.8 oz |
| | | | (45 g) | (50 g) |

Spray Properties: Aerosol Spray

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A product release system to atomize a cosmetic composition comprising:
(a) pressure-resistant packaging;
(b) a spray head containing a capillary; and
(c) a propellant-containing cosmetic composition, wherein the atomization is done using the capillary, and the composition is non-liquid at 25° C. or has a viscosity greater than 5,000 mPa s, measured at a temperature of 25° C. and a shear speed of 12.9 s⁻¹.

2. A product release system according to claim 1, wherein the capillary has a diameter of 0.1 to 1 mm and a length of 5 to 100 mm.

3. A product release system according to claim 1, wherein the spray rate is 0.01 to 5 g/s.

4. A product release system according to claim 1, wherein the propellants are selected from the group consisting of propane, butane, dimethyl ether, fluorinated hydrocarbons, and mixtures thereof.

5. A product release system according to claim 1, wherein the composition is a gel, wax, or emulsion.

6. A product release system according to claim 5, wherein the gel comprises at least one thickener or gel-former in a quantity of from 0.01 to 20 wt. %.

7. A product release system according to claim 6, wherein said thickener or gel-former is a thickened polymer, selected from the group consisting of copolymers of at least one first type of monomer, which is acrylic acid or methacrylic acid and at least one second type of monomer, which is an ester of acrylic acid or ethoxylated fatty alcohol, crosslinked polyacrylic acid, crosslinked copolymers of at least one first type of monomer, which is acrylic acid or methacrylic acid and at least one second type of monomer, which is selected from the group consisting of esters of acrylic acid with C10 to C30 alcohols, a copolymer of at least one first type of monomer which is acrylic acid or methacrylic acid and at least one second type of monomer which is an ester of itaconic acid or an ethoxylated fatty alcohol, copolymers of at least one first type of monomer, which is acrylic acid or methacrylic acid, at least one second type of monomer, which is an ester of itaconic acid or an ethoxylated C10 to C30 alcohol and a third type of monomer of from C1 to C4 aminoalkyl acrylates, copolymers of two or more monomers selected from the group consisting of acrylic acid, methacrylic acid, acrylic acid esters, and methacrylic acid esters, copolymers of vinyl pyrrolidone and ammonium acryloyl dimethyltaurate, copolymers of ammonium acryloyl dimethyltaurate and monomers of esters of methacrylic acid and ethoxylated fatty alcohols, hydroxyethyl cellulose, hydroxypropyl cellulose, hydroxypropyl guar, glyceryl polyacrylate, glycerylpoly methacrylate, copolymers of at least one C2, C3, or C4 alkylene and styrene, polyurethanes, hydroxypropyl starch phosphate, polyacrylamide, copolymer crosslinked with decadiene from maleic acid anhydride and methyl vinyl ether, locust bean gum, guar gum, xanthan, dehydroxanthan, carrageenan, karaya gum, hydrolyzed corn starch, copolymers of polyethylene oxide, fatty alcohols, and saturated methylene diphenyl diisocyanate.

8. A product release system according to claim 5, wherein the waxy composition comprises at least one wax that is solid at 25° C. in a quantity of from 10 to 80 wt. %.

9. A product release system according to claim 8, wherein the wax is selected from the group consisting of paraffin waxes, polyolefin waxes, wool wax, wool wax alcohols, candelilla wax, olive wax, carnauba wax, Japan wax, apple wax, hydrogenated fats, fatty acid esters, fatty acid glycerides, fatty acid triglycerides, polyethylene glycol waxes, silicone waxes and mixtures thereof.

10. A product release system according to claim 5, wherein the emulsion-type composition is selected from the group consisting of water-in-oil emulsions, oil-in-water emulsions, microemulsions, and wherein the emusion-type composition further comprises at least one emulsifier in a quantity of from 0.1 to 30 wt. %, and at least one oil in a quantity of from 1 to 20 wt. %, and water.

11. A product release system according to claim 10, wherein the oil is selected from the group consisting of silicone oils, mineral oils, isoparaffin oils, paraffin oils, squalane, sunflower seed oil, coconut oil, castor oil, lanolin oil, jojoba oil, corn oil, and soy oil.

12. A product release system according to claim 10, wherein the emulsifier is selected from the group consisting of addition products of 2 to 30 mol ethylene oxide, 1 to 5 mol propylene oxide to C8 to C22 fatty alcohols, addition products of 2 to 30 mol ethylene oxide, 1 to 5 mol propylene oxide to C12 to C22 fatty acids, addition products of 2 to 30 mol ethylene oxide, 1 to 5 mol propylene oxide to alkyl phenols with 8 to 15 C atoms in the alkyl group, C12 to C22 fatty acid monoesters and diesters of addition products of 1 to 30 mol ethylene oxide to glycerin, addition products of 5 to 60 mol ethylene oxide to castor oil or to hydrogenated castor oil, mono-, di-, or triesters of phosphoric acid with addition products of 2 to 30 mol ethylene oxide to C8 to C22 fatty alcohols, esters of saccharose and one or two C8 to C22 fatty acids, esters of sorbitan and one, two, or three C8 to C22 fatty acids and one ethoxylation level of 4 to 20, polyglyceryl fatty acid esters of one, two, or more C8 to C22 fatty acids and polyglycerol with 2 to 20 glyceryl units, alkylglycosides, C8-22 alkyldimethyl benzyl ammonium compounds, C8-22 alkyltrimethyl ammonium compounds, C8-22 alkyldimethyl hydroxyethyl ammonium compounds, di-(C8-22 alkyl)-dimethyl ammonium compounds, C8-22 alkylpyridinium salts, C8-22 alkylamido ethyl trimethyl ammonium ether sulfates, C8-22 alkylmethyl amine oxides, C8-22 alkyl amino ethyl dimethyl amine oxides, amidoamines, and quaternized amidoamines.

13. A product release system according to claim 1, wherein the composition comprises at least one additional active ingredient or additive, selected from the group consisting of hair-conditioning materials, hair-setting materials, silicone compounds, photoprotective materials, preservatives, pigments, direct-penetrating hair dyes, particle-shaped materials, oxidizing agents, reducing agents, oxidative hair dye precursor products and mixtures thereof.

14. A product release system according to claim 13, wherein said active ingredients or additives are contained in a quantity of from 0.01 to 20 wt. %.

15. A product release system according to claim 13, wherein a polymer with anionic groups or groups that can be anionized is contained as said hair-conditioning or said hair-setting material, with said polymer being selected from the group consisting of terpolymers of acrylic acid, ethyl acrylate, and N-tert-butylacrylamide, crosslinked or uncrosslinked vinyl acetate/crotonic acid copolymers, terpolymers selected from the group consisting of tert.-butylacrylate, ethyl acrylate, and methacrylic acid, sodium polystyrene sulfonate, copolymers of vinyl acetate, crotonic acid, and vinyl propionate, copolymers selected from the group consisting of vinyl acetate, crotonic acid, and vinyl neodecanoate, aminomethyl propanol acrylate copolymers, copolymers of vinyl pyrrolidone and at least one additional monomer selected from the group consisting of acrylic acid, methacrylic acid, acrylic acid esters, and methacrylic acid esters, copolymers of methyl vinyl ether and maleic acid monoalkyl esters, aminomethyl propanol salts of copolymers of allylmethacrylate and at least one additional monomer selected from the group consisting of acrylic acid, methacrylic acid, acrylic acid esters, and methacrylic acid esters, crosslinked copolymers of ethyl acrylate and methacrylic acid, copolymers selected from the group consisting of vinyl acetate, mono-n-butyl maleate, and isobomyl acrylate, copolymers of two or more monomers selected from the group consisting of acrylic acid, methacrylic acid, acrylic acid esters, and methacrylic acid esters, copolymers of octylacrylamide and at least one monomer selected from the group consisting of acrylic acid, methacrylic acid, acrylic acid esters, and methacrylic acid esters, polyesters of diglycol, cyclohexanedimethanol, isophthalic acid, and sulfoisophthalic acid.

16. A product release system according to claim 13, wherein a polymer with cationic groups or groups that can be cationized is contained as said hair-conditioning or as said hair-setting material, with said polymer being selected from the group consisting of cationic cellulose derivatives of hydroxyethyl cellulose and diallyl dimethyl ammonium chloride, cationic cellulose derivatives of hydroxyethyl cellulose and epoxide substituted with trimethyl ammonium, poly(dimethyldiallyl ammonium chloride), copolymers of acrylamide and dimethyldiallyl ammonium chloride, quaternary ammonium polymers, formed by the reaction of diethylsulfate and a copolymer of vinyl pyrrolidone and dimethylaminoethyl methacrylate, quaternary ammonium polymers from methylvinylimidazolium chloride and vinyl pyrrolidone, Polyquatemium-35, a polymer of trimethyl ammonium ethyl methacrylate chloride, Polyquatemium-57, dimethylpolysiloxane terminally substituted with quaternary ammonium groups, a copolymer of vinyl pyrrolidone, dimethylaminopropyl methacrylamide, and methacryloylamino propyl lauryl dimethyl ammonium chloride, chitosan and salts thereof, hydroxyalkyl chitosans and salts thereof, alkyl hydroxyalkyl chitosans and salts thereof, N-hydroxyalkyl chitosan alkyl ether, a copolymer of vinyl caprolactam, vinyl pyrrolidone, and dimethylaminoethyl methacrylate, copolymers of vinyl pyrrolidone and dimethylaminoethyl methacrylate, copolymers of vinyl pyrrolidone, vinyl caprolactam, and dimethylaminopropylacrylamide, poly- or oligo-esters, constructed from at least one first type of monomer, which is a hydroxycarboxylic acid substituted with at least one quaternary ammonium group.

17. A product release system according to claim 13, wherein a zwitterionic and/or an amphoteric polymer is contained as said hair-conditioning or as said hair-setting material, with said polymer being selected from the group consisting of copolymers of octylacrylamide, acrylic acid, butylaminoethyl methacrylate, methyl methacrylate, and hydroxypropyl methacrylate, copolymers of lauryl acrylate, stearyl acrylate, ethylamine oxide methacrylate, and at least one monomer selected from the group consisting of acrylic acid, methacrylic acid, acrylic acid esters, and methacrylic acid esters, copolymers of methacryloyl ethyl betaine and at least one monomer of methacrylic acid and methacrylic acid esters, copolymers of acrylic acid, methylacrylate, and methacrylamide propyl trimethylammonium chloride, oligomers or polymers that can be produced from quaternary crotonic betaines or quaternary crotonic betaine esters.

18. A product release system according to claim 13, wherein a nonionic polymer is contained as said hair-conditioning or as said hair-setting material, with said polymer being selected from the group consisting of polyvinylpyrrolidone, polyvinyl caprolactam, vinyl pyrrolidone/vinylacetate copolymers, polyvinylalcohol, isobutylene/ethyl-maleimide/hydroxyethylmaleimide copolymer, copolymers of vinyl pyrrolidone, vinyl acetate, vinyl propionate and mixtures thereof.

19. A product release system according to claim 13, wherein at least one silicone compound is contained, which is selected from the group consisting of cyclic dimethylsiloxanes, linear polydimethylsiloxanes, block polymers of polydimethylsiloxane, and polyethylene oxide and polypropylene oxide, polydimethylsiloxanes with terminal or lateral polyethylene oxide or polypropylene oxide radicals, polydimethylsiloxanes with terminal hydroxyl groups, phenyl-substituted polydimethylsiloxanes, silicone emulsions, silicone elastomers, silicone waxes, silicone gums, amino-substituted silicones, and silicones substituted with one or more quaternary ammonium groups.

20. A product release system according to claim 13, wherein at least one photoprotective material is contained, which is selected from the group consisting of 4-methoxy cinnamic acid-2-ethylhexyl ester, methyl methoxy cinnamate, 2-hydroxy-4-methoxy benzophenone-5-sulfonic acid, polyethoxylated p-aminobenzoates and mixtures thereof.

21. A product release system according to claim 13, wherein an active ingredient and additive is contained, which is selected from the group consisting of betaine, panthenol, panthenyl ethyl ether, sorbitol, protein hydrolysates, plant extracts, A-B block copolymers of alkyl acrylates and alkyl methacrylates, A-B block copolymers of alkyl methacrylates and acrylonitrile, A-B-A block copolymers of lactide and ethylene oxide, A-B-A block copolymers of caprolacton and ethylene oxide, A-B-C block copolymers of alkylene or alkadiene compounds, styrene and alkyl methacrylates, A-B-C block copolymers of acrylic acid, styrene, and alkyl methacrylates, star-shaped block copolymers, hyper-branched polymers, dendrimers, intrinsically electrically conducting 3,4-polyethylene dioxythiophenes, and intrinsically electrically conducting polyanilines.

22. A product release system according to claim 13, wherein at least one pigment is contained, which is selected from the group consisting of titanium dioxide, black iron oxide, yellow iron oxide , red and brown iron oxide, manganese violet, ultramarine, chromium oxide hydrate, iron blue, bismuth oxichloride, carmine (cochineal), pearl shine and color pigments based on mica which are coated with a metallic oxide or a metallic oxychloride such as titanium dioxide or bismuth oxychloride as well as, if necessary, additional color-providing materials such as iron oxides, iron blue, ultramarine, or carmine, and wherein the color is determined by varying the layer thickness.

23. A product release system according to claim 13, wherein at least one particle-shaped material is contained, which is selected from the group consisting of silica, silicates, aluminates, alumina, mica, insoluble metallic salts, metallic oxides, minerals, insoluble polymer particles and mixtures thereof.

24. A product release system according to claim 1, wherein the composition is gel-like, has a viscosity of at least 5,000 mPa s, measured at a temperature of 25° C., has a shear speed of 12.9 s⁻¹, and comprises:
(a) 0.1 to 10 wt. % of at least one thickened polymer, selected from the group consisting of copolymers of at least one first type of monomer, which is acrylic acid or methacrylic acid and at least one second type of monomer, which is an ester of acrylic acid or an ethoxylated fatty alcohol, crosslinked polyacrylic acid, crosslinked copolymers of at least one first type of monomer, which is acrylic acid or methacrylic acid and at least one second type of monomer, which is an ester of acrylic acid with C10 to C30 alcohols, copolymers of at least one first type of monomer, which is acrylic acid or methacrylic acid and at least one second type of monomer, which is an ester of itaconic acid or ethoxylated fatty alcohol, copolymers of at least one first type of monomer, which is acrylic acid or methacrylic acid, at least one second type of monomer, which is an ester of itaconic acid or ethoxylated C10 to C30 alcohol and a third type of monomer, which is a C1 to C4 aminoalkyl acrylate, copolymers of two or more monomers, selected from the group consisting of acrylic acid, methacrylic acid, acrylic acid esters, and methacrylic acid esters, copolymers of vinyl pyrrolidone and ammonium acryloyl dimethyltaurate, copolymers of ammonium acryloyl dimethyltaurate and monomers of esters of methacrylic acid or ethoxylated fatty alcohols, hydroxyethyl cellulose, hydroxypropyl cellulose, hydroxypropyl guar, glyceryl polyacrylate, glyceryl polymethacrylate, copolymers of at least one C2, C3, or C4 alkylene and styrene, polyurethanes, hydroxypropyl starch phosphate, polyacrylamide, copolymer crosslinked with decadiene from maleic acid anhydride and methyl vinyl ether, locust bean gum, guar gum, xanthan, dehydroxanthan, carrageenan, karaya gum, hydrolyzed corn starch, copolymers of polyethylene oxide, fatty alcohols and saturated methylene diphenyl diisocyanate; and
(b) 0.1 to 5 wt. % of at least one hair-setting or hair-conditioning polymer.

25. A product release system according to claim 1, wherein the composition is present in the form of an oil-in-water emulsion, a water-in-oil emulsion, or a microemulsion, it has a viscosity of at least 0.0007 lb/in² s (5,000 mPa s), (measured at a temperature of 77° F. (25° C.)) and a shear speed of 12.9 s⁻¹, and it contains
(a) 1 to 20 wt. % of at least one oil or wax, selected from the group consisting of silicone oils, mineral oils, isoparaffin oils, paraffin oils, squalane, plant oils, paraffin waxes, polyolefin waxes, wool wax, wool wax alcohols, candelilla wax, olive wax, carnauba wax, Japan wax, apple wax, hydrogenated fats, fatty acid esters, fatty acid glycerides, fatty acid triglycerides, polyethylene glycol waxes, silicone waxes and mixtures thereof, and
(b) 0.01 to 30 wt. % of at least one emulsifier.
